(19) United States
(12) Patent Application Publication
(10) Pub. No.: US 2008/0213177 A1
(43) Pub. Date: Sep. 4, 2008

(54) Nanoparticles Comprising Rna Ligands

(76) Inventors: Thomas William Rademacher, Oxford (GB); Manuel Martin-Lomas, Madrid (ES); Soledad Penades, Madrid (ES); Rafael Ojeda, Madrid (ES); African G. Barrientes, Madrid (ES); Khalid Gumaa, Oxford (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/597,215

(22) PCT Filed: May 24, 2005
(86) PCT No.: PCT/GB2005/002058

§ 371 Date: Dec. 18, 2007

Related U.S. Application Data
(60) Provisional application No. 60/573,805, filed on May 24, 2004.

(30) Foreign Application Priority Data
May 24, 2004 (GB) 0411537.4

(51) Int. Cl. A61K 9/14; C07H 21/02; C07F 19/00; A61K 51/04; A61K 9/12; A61P 11/00; A61K 49/00; A61K 31/7088; C12N 15/09

(52) U.S. Cl. 424/1.73; 536/23.1; 536/24.1; 534/15; 435/440; 424/489; 514/44; 424/9.1; 424/45

(57) ABSTRACT

Materials and methods are provided for making nanoparticles having a core including metal and/or semiconductor atoms, which core is covalently linked to a plurality of ligands comprising a RNA ligand. The RNA ligands may include siRNA or miRNA. Also provided are uses of these nanoparticles in therapy and diagnosis.

### FIELD OF THE INVENTION

The present invention relates to nanoparticles, and more particularly to nanoparticles comprising RNA ligands such as small interfering RNA (siRNA) and micro RNA (miRNA), and their use in a range of applications.

### BACKGROUND OF THE INVENTION

Small RNA molecules have been found to play multiple roles in regulating gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing. RNAi activity limits transposon mobilization and provides an antiviral defence (Pal-Bhadra et al, 2004). A role for the RNAi machinery and small RNAs in targeting of heterochromatin complexes and epigenetic gene silencing at specific chromosomal loci has also been demonstrated (Verdel et al, 2004). Double-stranded RNA (dsRNA)-dependent post transcriptional silencing, also known as small inhibitory RNA (siRNA) or RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response (Elbashir et al, 2001). The decrease in expression of targeted gene products can be extensive with 90% silencing induced by a few molecules of siRNA.

Since the delivery of small-molecule oligonucleotides can bypass the difficulties associated with gene therapy, the use of siRNA may have advantages over traditional gene therapy. To date, efficient delivery of vector-based therapeutic genes in vivo remains an obstacle to successful gene therapy. It has been observed that although knockdown of the target gene by siRNA is not permanent, a single siRNA transfection can lead to a prolonged inhibition of the target protein in the parent as well as progeny cells (Tuschl, 2001). However, there is still a problem in the art in delivery of siRNA.

WO 02/32404 (Consejo Superior de Investigaciones Scientificas) discloses nanoparticles formed from metal or semiconductor atoms in which ligands comprising carbohydrates are covalently linked to the core of the nanoparticles. These nanoparticles are used for modulating carbohydrate mediated interactions and are soluble and non-toxic. PCT application claiming priority from GB-A-0313259.4 (Consejo Superior de Investigaciones Scientificas and Midatech Limited) discloses magnetic nanoparticles having cores comprising passive and magnetic metal atoms, the core being covalently linked to ligands.

### SUMMARY OF THE INVENTION

Broadly, the present invention relates to nanoparticles comprise having a core including metal and/or semiconductor atoms, the core being linked to RNA ligands. The RNA ligands are typically short RNA sequences designed to mimic small interfering RNA (siRNA) and micro-RNA sequences (miRNA). The nanoparticles can be used to deliver the RNA ligands and have applications in a wide range of applications, in in vitro systems and for therapeutic or diagnostic applications. By way of example, the nanoparticles of the present invention may be employed (1) for targeted transcriptional gene silencing, (2) for targeted mRNA degradation, (3) for imaging mRNA, (4) for inhibiting pathways by employing a plurality of RNA ligands on the same or different nanoparticles, (5) for aerosol delivery, e.g. to the lungs, (6) in combination with mRNA silencing for targeting siRNA resistant mRNA and (7) for use a tool in functional genomics.

In the art, short RNA sequences are termed “short interfering RNAs” (siRNAs) or “microRNAs” (miRNAs) depending in their origin. Both types of sequence may be used to down-regulate gene expression by binding to complimentary RNAs (nmRNA) and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences. The RNAi pathway also acts on the genome as discussed in Science, 301: 1060-1061, 2003).

The RNA associated with the nanoparticles may be single stranded or double stranded (duplex). Where miRNA-like sequences are used as ligands, the RNA sequences may be hairpins, that is include partially complementary regions towards their ends that can anneal to form the hairpin. The nanoparticles may optionally comprise further types of ligands, such as carbohydrates to form glyconanoparticles, and/or more than one species of siRNA. The nanoparticles and their uses are discussed in more detail below. Advantageously, the attachment of the siRNA to the nanoparticle may provide protection for the siRNA from exoribonucleases present in the blood, tissue culture media or within cells.

Accordingly, in a first aspect, the present invention provides a nanoparticle which comprises a core including metal and/or semiconductor atoms, wherein the core is covalently linked to a plurality of ligands and the ligand comprise a RNA ligand.

The siRNA ligand forming the nanoparticles may be single stranded or double stranded (duplex). However, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and, preferably when the nanoparticles are administered in vivo, that the siRNA is short enough to reduce a host response.

miRNA ligands are typically single stranded and have regions that are partially complementary enabling the ligands to form a hairpin. miRNA is typically in a form of single-stranded RNA and is thought to regulate the expression of other genes. miRNAs are RNA genes which are transcribed from DNA, but are not translated into protein. A DNA sequence that codes for an miRNA gene is longer than the miRNA. This DNA sequence includes the miRNA sequence and an approximate reverse complement.

When this DNA sequence is transcribed into a single-stranded RNA molecule, the miRNA sequence and its reverse-complement base pair to form a double stranded RNA segment; overall this RNA structure is called a hairpin structure (‘short hairpin RNA’ or shRNA). The Dicer enzyme then cuts double stranded region out of the hairpin structure, to release the mature miRNA.

shRNAs can be produced within a cell by transfecting the cell with a DNA construct encoding the shRNA sequence under control of a RNA polymerase III promoter, such as the human H1 or 7SK promoter. Alternatively, the shRNA may be synthesised exogenously and introduced directly into the cell.

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double stranded siRNA, the molecule may have symmetric 3′ overhangs, e.g. of one or two (ribo)nucleotides, typically a UU of dTdT 3′ overhang.

Where miRNAs are produced by cleavage of shRNA, the shRNA sequence is preferably between 40 and 100 bases in length, more preferably between 40 and 70 bases in length. The stem of the hairpin is preferably between 19 and 30 base pairs in length. The stem may contain G-U pairings to stabilise the hairpin structure.

In embodiments using double stranded RNA, the sense and antisense strands may be annealed to form a duplex. By including the duplex in the reaction mixture to form the nanoparticles, the RNA can attach to the core during self-assembly of the particles. Depending on the number of derivatised ends of the duplex siRNA (four possible, 5′ and 3′ ends of each strand), up to four nanoparticles may become attached to a single siRNA duplex, and theoretically forming up to fifteen possible constructs for each duplex siRNA, with one of these having four nanoparticles (two at each end), four with one nanoparticle and six with two nanoparticles and four with three nanoparticles. Where single stranded siRNA is employed, nanoparticles core may become attached to either or both derivatised ends (i.e. 5′ or 3′ ends) of the siRNA, e.g. producing three different species of nanoparticle. These nanoparticles may be used in this form or the method may optionally comprise the further step of annealing siRNA containing nanoparticle to a complementary strand of siRNA, thereby forming a duplex on the preformed nanoparticle in situ. In the formation of miRNA-like ligands, one or two nanoparticles typically become attached to the ends of the RNA sequence.

Accordingly, in a further aspect, the present invention provides a method of producing a nanoparticle as described herein. Conveniently, the method comprises conjugating the RNA ligands to the core of the nanoparticle by derivatising the strand(s) of RNA with a linker and including the derivatised RNA in a reaction mixture from which the core of the nanoparticle is synthesised. During self-assembly of the nanoparticles, the nanoparticle cores attach to the RNA via the linker. Preferably, the linker is a disulphide linker, for example a mixed disulphide linker, although ethylene linkers or peptide linkers may also be employed. An exemplary linker group is represented by the general formula HO—(CH₂)ₙ—S—S—(CH₂)ₘ—OH wherein n and m are independently between 1 and 5. The RNA can conveniently be linked to the spacer via a terminal phosphate group, and in the case of the preferred mixed disulphide linkers via one of the terminal hydroxyl groups. When the nanoparticles are synthesized, the —S—S— of the linker splits to form two thio linkers that can each covalently attach to the core of the nanoparticle via a —S— group. The use of mixed disulphide linker helps to avoid the formation of RNA dimers.

As indicated above, where the RNA linked to the nanoparticle core is single stranded, the method may comprise the further step of annealing a RNA molecule which is complementary to the first strand to provide a double stranded RNA ligand attached to the nanoparticle. It is also possible to prepare nanoparticles with annealed double stranded RNA, with one or both of the strands functionalised with the disulphide linker. Alternatively or additionally, the sense and antisense strand of the RNA may linked to different nanoparticles and annealed together.

In a preferred embodiment of the invention, in order for the duplex RNA to be incorporated in the self-assembling nanoparticle, one or both of the ends of the RNA may be derivatised with a mixed disulphide. Following incorporation of the duplex into the nanoparticle, both the RNA and the chemical constituents of the mixed disulphide will be incorporated into the bead. The chemical composition, therefore, of the mixed disulphide can contain important information in its own right, such as targeting properties (e.g. a member of a specific binding pair) or possibly provide further physical characteristics to the final formed nanoparticle. The mixed disulphide can be attached to either the 3′ end (or ends) or the 5′ end (or ends) of the sense or anti-sense strands.

In one embodiment, nanoparticles having cores comprising gold atoms may be synthesised using the protocol first described in WO 02/32404 in which disulphide linkers are employed to derivatise the ligands and the derivatised ligands are reacted with HAuCl₄ (tetrachloroauric acid) in the presence of reducing agent to produce the nanoparticles. On this method, the disulphide protected RNA in methanol or water may be added to an aqueous solution of tetrachloroauric acid. A preferred reducing agent is sodium borohydride. These and other features of the method are described WO 02/32404.

In some applications, a plurality of different RNA molecules may be employed. These may be provided as different ligands conjugated to one set of nanoparticles or the different RNA molecules may be separate populations of nanoparticles, and optionally mixed together. In the first case, the skilled person will appreciate that where a mixture of products results from the conjugation of the RNA to the nanoparticles, a set of nanoparticles may comprise a range of products as indicated above. Where a plurality of ligands are employed, ligands mimicking both siRNA and miRNA may be employed.

In addition to the RNA molecule(s), the nanoparticles may comprise one or more further types of ligands and/or the RNA ligands may comprise one or more different types of groups or domains in addition to the RNA component. For example, the additional ligands, or groups or domains of ligands, may include one or more peptide, a protein domain, a nucleic acid molecule, a lipidic group, a carbohydrate group, any organic or anionic or cationic group. The carbohydrate group may be a polysaccharide, an oligosaccharide or a monosaccharide group. Preferred ligands include glycoconjugates, thereby forming glyconanoparticles. As indicated below in the discussion of uses of the nanoparticles, the ligands (RNA or further ligands) may be members of a specific binding pair and used for targeting the nanoparticles to a target location where the other member of the specific binding pair is present. Where a nucleic acid molecule is present in addition to the RNA ligand(s), the nucleic acid molecule may comprise single or double stranded DNA or RNA. The particles may have more than one species of ligand immobilised thereon, e.g. 2, 3, 4, 5, 10, 20 or 100 different ligands. Alternatively or additionally, a plurality of different types of nanoparticles may be employed together. In preferred embodiments, the mean number of total ligands linked to an individual metallic core of the particle is at least one ligand, more preferably 50 ligands, and most preferably 60 ligands.

Preferably, the nanoparticles have cores having mean diameters between 0.5 and 50 nm, more preferably between 0.5 and 10 nm, more preferably between 1.0 and 5 nm, and still more preferably between 3.0 and 7.0 nm. When the ligands are considered in addition to the cores, preferably the overall mean diameter of the particles is between 5.0 and 100 nm, more preferably between 5 and 50 nm and most preferably between 10 and 30 nm. The mean diameter can be measured using techniques well known in the art such as transmission electron microscopy.

The core material can be a metal or semiconductor and may be formed of more than one type of atom. Preferably, the core material is a metal selected from Au, Fe or Cu. Nanoparticle cores may also be formed from alloys including Au/Fe, Au/Cu, Au/Gd, Au/Fe/Cu, Au/Fe/Gd and Au/Fe/Cu/Gd, and may be used in the present invention. Preferred core materials are Au and Fe, with the most preferred material being Au. The cores of the nanoparticles preferably comprise between about 100 and 500 atoms (e.g. gold atoms) to provide core diameters in the nanometre range. Other particularly useful core materials are doped with one or more atoms that are NMR active, allowing the nanoparticles to be detected using NMR, both in vitro and in vivo. Examples of NMR active atoms include Mn⁺², Gd⁺³, Eu⁺², Cu⁺², V⁺², Co⁺², Ni⁺², Fe⁺², Fe⁺³ and lanthanides⁺³, or the quantum dots described elsewhere in this application.

Nanoparticle cores comprising semiconductor atoms can be detected as nanometre scale semiconductor crystals are capable of acting as quantum dots, that is they can absorb light thereby exciting electrons in the materials to higher energy levels, subsequently releasing photons of light at frequencies characteristic of the material. An example of a semiconductor core material is cadmium selenide, cadmium sulphide, cadmium tellurium. Also included are the zinc compounds such as zinc sulphide.

In some embodiments, the nanoparticle of the present invention or RNA molecule comprises a detectable label. The label may be an element of the core of the nanoparticle or the RNA ligand or another ligand. The label may be detectable because of an intrinsic property of that element of the nanoparticle or by being linked, conjugated or associated with a further moiety that is detectable. Preferred examples of labels include a label which is a fluorescent group, a radionuclide, a magnetic label or a dye. Fluorescent groups include fluorescein, rhodamine or tetramethyl rhodamine, Texas-Red, Cy3, Cy5, etc., and may be detected by excitation of the fluorescent label and detection of the emitted light using Raman scattering spectroscopy (Y. C. Cao, R. Jin, C. A. Mirkin, Science 2002, 297: 1536-1539).

In some embodiments, the nanoparticles may comprise a radionuclide for use in detecting the nanoparticle using the radioactivity emitted by the radionuclide, e.g. by using PET,SPECT, or for therapy, i.e. for killing target cells. Examples of radionuclides commonly used in the art that could be readily adapted for use in the present invention include ^{99m}Tc, which exists in a variety of oxidation states although the most stable is TcO⁴⁻; ³²P or ³³P; ⁵⁷Co; ⁵⁹Fe; ⁶⁷Cu which is often used as Cu²⁺ salts; ⁶⁷Ga which is commonly used a Ga³⁺ salt, e.g. gallium citrate; ⁶⁸Ge; ⁸²Sr; ⁹⁹Mo; ¹⁰³Pd; ¹¹¹In which is generally used as In³⁺ salts; ¹²⁵I or ¹³¹I which is generally used as sodium iodide; ¹³⁷Cs; ¹⁵³Gd; ¹⁵³Sm; ¹⁵³Au; ¹⁸⁶Re; ²⁰¹Tl generally used as a Tl⁺ salt such as thallium chloride; ³⁹Y³⁺; ⁷¹Lu³⁺; and ²⁴Cr²⁺. The general use of radionuclides as labels and tracers is well known in the art and could readily be adapted by the skilled person for use in the aspects of the present invention. The radionuclides may be employed most easily by doping the cores of the nanoparticles or including them as labels present as part of ligands immobilised on the nanoparticles.

Additionally or alternatively, the nanoparticles of the present invention, or the results of their interactions with other species, can be detected using a number of techniques well known in the art using a label associated with the nanoparticle as indicated above or by employing a property of them. These methods of detecting nanoparticles can range from detecting the aggregation that results when the nanoparticles bind to another species, e.g. by simple visual inspection or by using light scattering (transmittance of a solution containing the nanoparticles), to using sophisticated techniques such as transmission electron microscopy (TEM) or atomic force microscopy (AFM) to visualise the nanoparticles. A further method of detecting metal particles is to employ plasmon resonance that is the excitation of electrons at the surface of a metal, usually caused by optical radiation. The phenomenon of surface plasmon resonance (SPR) exists at the interface of a metal (such as Ag or Au) and a dielectric material such as air or water. As changes in SPR occur as analytes bind to the ligand immobilised on the surface of a nanoparticle changing the refractive index of the interface. A further advantage of SPR is that it can be used to monitor real time interactions. As mentioned above, if the nanoparticles include or are doped with atoms which are NMR active, then this technique can be used to detect the particles, both in vitro or in vivo, using techniques well known in the art. Nanoparticles can also be detected using a system based on quantitative signal amplification using the nanoparticle-promoted reduction of silver (I). Fluorescence spectroscopy can be used if the nanoparticles include ligands as fluorescent probes. Also, isotopic labelling of the carbohydrate can be used to facilitate their detection.

The present invention provides a way of presenting a spherical array of ligands having advantages over other types of array proposed in the prior art. In particular, the nanoparticles are soluble in most organic solvents and especially water. This can be used in their purification and importantly means that they can be used in solution for presenting the ligand immobilised on the surface of the particle. The fact that the nanoparticles are soluble has the advantage of presenting the ligands in a natural conformation. For therapeutic applications, the nanoparticles are non toxic, soluble and stable under physiological conditions.

In some embodiments, the core of the nanoparticles may be magnetic and comprise magnetic metal atoms, optionally in combination with passive metal atoms. By way of example, the passive metal may be gold, platinum, silver or copper, and the magnetic metal may be iron or gadolinium. In preferred embodiments, the passive metal is gold and the magnetic metal is iron. In this case, conveniently the ratio of passive metal atoms to magnetic metal atoms in the core is between about 5:0.1 and about 2:5. More preferably, the ratio is between about 5:0.1 and about 5:1. As used herein, the term “passive metals” refers to metals which do not show magnetic properties and are chemically stable to oxidation. The passive metals of the invention may be diamagnetic. Diamagnetic refers to materials with all electrons paired which thus have no permanent net magnetic moment per atom. Magnetic materials have some unpaired electrons and are positively susceptible to external magnetic fields—that is, the external magnetic field induces the electrons to line up with the applied field, so the magnetic moments of the electrons are aligned. Magnetic materials may be paramagnetic, superparamagnetic or ferromagnetic. Paramagnetic materials are not very susceptible to external magnetic fields and do not retain their magnetic properties when the external magnetic field is removed. Ferromagnetic materials are highly susceptible to external magnetic fields and contain magnetic domains even when no external magnetic field is present because neighbouring atoms cooperate so their electron spins are parallel. External magnetic fields align the magnetic moments of neighbouring domains, magnifying the magnetic affect. Very small particles of materials that normally have ferromagnetic properties are not ferromagnetic, as the cooperative effect does not occur in particles of 300 nm or less so the material has no permanent magnetism. However, the particles are still very susceptible to external magnetic fields and have strong paramagnetic properties, and are known as superparamagnetic. Preferably, the nanoparticles of the invention are superparamagnetic.

Examples of nanoparticles which have cores comprising a paramagnetic metal, include those comprising Mn⁺², Gd⁺³, Eu⁺², Cu⁺², V⁺², Co⁺², Ni⁺², Fe⁺², Fe⁺³ and lanthanides⁺³.

Other magnetic nanoparticles may be formed from materials such as MnFe (spinel ferrite) or CoFe (cobalt ferrite) can be formed into nanoparticles (magnetic fluid, with or without the addition of a further core material as defined above. Examples of the self-assembly attachment chemistry for producing such nanoparticles is given in Biotechnol. Prog., 19:1095-100 (2003), J. Am. Chem. Soc. 125:9828-33 (2003), J. Colloid Interface Sci. 255:293-8 (2002).

In a further aspect, the present invention provides compositions comprising populations of one or more of the above defined particles. In some embodiments, the populations of nanoparticles may have different densities of the same or different ligands attached to the core. In some cases, it may be desirable to encapsulate the nanoparticles to enable the delivery of a plurality of nanoparticles to a target site. Suitable encapsulation technologies are well known the those skilled in the art. The encapsulated population of nanoparticles may be of one, two, three or a plurality of different types. In one embodiment, the present invention provides an aerosol composition of nanoparticles as defined herein. The aerosol composition may comprise the nanoparticles and optionally a diluent. Examples of the uses of these compositions are discussed below.

The following examples of application for the nanoparticles are provided by way of illustration and not limitation to support the wide applicability of the technologies described herein. A review of the general uses of siRNA is provided in Dorseet & Tuschl, Nature Reviews, 3: 318-329, 2004.

In a further aspect, the present invention provides the above defined nanoparticles for use in therapy or diagnosis.

In a further aspect, the present invention provides the use of the above defined nanoparticles for the preparation of a medicament for the treatment of a condition ameliorated by the administration of the nanoparticles. Examples of specific uses that may be treated according to the present invention are described below, along with other applications of the nanoparticles, both in vitro and in vivo uses. For example, the nanoparticles described herein or their derivatives can be formulated in pharmaceutical compositions, and administered to patients in a variety of forms, in particular to treat conditions ameliorated by the administration of a RNA ligand. By way of example, this may be used for the treatment of a condition ameliorated by the down regulation of expression of a gene by the RNA, wherein the gene is down regulated by the RNA, or for the treatment of a condition associated with over expression of a gene which is targeted and down regulated by the RNA.

Regulation of Gene Expression

Nanoparticles comprising RNA ligands may be used to regulate gene expression in a number of ways including targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

Generally speaking, the present invention provides the use of nanoparticles as described herein for down regulating a target gene. In this application, the down regulation may be in vitro, for example to study gene expression, or in vivo, either in an experimental system of interest or for medical use, that is the nanoparticles may be employed for the preparation of a medicament for the treatment of a condition which is ameliorated by the down regulation of expression of the target gene by the RNA or for the treatment of a condition associated with over expression of a target gene. By way of example, the conditions may include cancer, e.g. breast cancer which may be treated using RNA based on Her2/Neu sequences. As indicated herein, as the down regulation provided by RNA may be transient in effect, in some embodiments it is preferred that the nanoparticles further comprise a radionuclide, drug or other agent for treating or killing the cells in which the nanoparticles down regulate the target genes.

RNA interference can be used to treat any disease that is linked to an overactive gene or genes, for example most forms of cancer, e.g. using the RNA for oncogene suppression. The shutting down of specific genes such as the cell death receptor in hepatitis or other disorders where overactive gene expression contributes to the disease pathology are also targets for RNA therapy. A further example of a suitable conditions for treatment using nanoparticles of the present invention are macular degeneration in the eye, or by applying RNA in its natural role as a means of combating pathogenic viruses by disabling their RNA, including HIV, hepatitis C or influenza, among others (Check, 2003; Zamore et al, 2003; Song et al, 2003; Matzke & Matzke, 2003).

Down Regulation of Pathways

It is particularly notable that the present invention allows more than one RNA molecule to be delivered, something which has not been previously possible in the art. Accordingly, in some embodiments, the present invention provides a nanoparticle composition having at least two different RNA sequences, either conjugated to the same nanoparticles or present in a composition of at least two different types of nanoparticles can be used to down regulate expression in gene pathways. The number of RNA ligands present in the composition will depend on the complexity of the pathway and the number of genes that need to be targeted for down regulation. Examples of pathways that might be targeted, either for study or treatment, include inflammatory pathways, anti-viral pathways, signalling pathways in cancer, metastasis pathways or metabolic pathways. By way of example, this includes the modulation of the neoglucgogenesis pathways for glucose production in type II diabetes.

In a further related embodiment, the RNA-nanoparticles can be used to down regulate the expression of a gene family by designing the RNA to target a domain conserved between family members.

In any of the uses of the nanoparticles for inhibiting gene expression using RNA, the nanoparticles may also comprise siRNA sequences and mRNA silencing sequences to target mRNA. These nanoparticles may be used to inhibit siRNA resistant mRNA. This may be used in situations where target cells are resistant to siRNA silencing because they express proteins that block the siRNA inhibiting machinery. In this case, siRNA resistance can be ameliorated by directing siRNA against the gene products which are expressed to induce the resistance.

Targeting Applications Using Additional Ligands or Using the RNA

In one application, the compositions of the nanoparticles of the invention can be used to impart targeting characteristics for the delivery of the RNA to target cells. This can be achieved by providing the nanoparticles with further types of ligands conjugated to the core of the nanoparticle or domains associated with the RNA ligands that enable the RNA nanoparticles to specifically interact with a target population of cells. By way of example, the RNA-containing nanoparticles can be preferentially directed to a population of cells by providing the nanoparticles with a ligand that is a member of a specific binding pair that is capable of specifically binding to its binding partner present on the surface of or inside of the target cells, e.g. to target a particular cell structure such as the nucleus. Examples of specific binding pairs suitable for use as ligands conjugated to the nanoparticles for targeting them include ligands and receptors, and many alternatives will be apparent to the skilled person. For example, a glucose derivatised nanoparticle may be used to target cells containing members of the GLUT family of proteins (18). Other glycoligands for the nanoparticle can be used such as Glcβ4GlcNAc or Glcβ4GlcNH₂. The former can be used to target the siRNA containing nanoparticle first to the cell surface GLUT transport proteins, and then upon entry into the cell (following cleavage by glucosidase), the GlcNAc will further target the siRNA nanoparticle to the nucleus. The latter construct will add positive charge to the surface of the nanoparticle, further facilitating adhesion and uptake into a cell. Since self-assembling nanoparticles can be used to incorporate heterogeneous ligands such as lipids, peptides or any other chemical constituent (e.g. as described above in the discussion of ligands) which is linked by a spacer to a disulphide, a wide variety of other targeting molecules can be used to target the RNA nanoparticles to a particular cell type. By way of example, these techniques may be used to target the nanoparticles carrying RNA to tumour cells.

In a further example of the use of the nanoparticles for targeting cell types, the RNA ligands of the nanoparticles may be used as the entity that provides the targeting of the nanoparticles, by directing the nanoparticles to cells in which mRNA that interacts with the RNA ligands is expressed. Examples of types of target cells that can be targeted in this way are tumour cells or virally infected cells, by using RNA to target the expression of viral genes or tumour markers or oncogenes in the target cells. In this embodiment, it is preferred that the RNA-nanoparticles are capable of permeating through the cell membrane, an effect that is provided by their small size and can optionally be enhanced by derivatising the nanoparticles with a membrane translocation signal (see Nature Biotechnology 18: 410-414, 2000). The targeting of the RNA-nanoparticles to cells in which the corresponding mRNA is expressed has the advantage of down regulating the target mRNA in a cell selective manner. However, as this effect is generally transient, it is preferred to provide the nanoparticle with a radionuclide or drug, so that the cells targeted using the RNA are selectively killed. The target cells and the treatment process can be imaged and followed by labelling the nanoparticles, for example as described above using radioactive or magnetic nanoparticles.

Imaging mRNA

In a further aspect, the present invention provides a method for detecting and/or imaging mRNA, which employs the nanoparticles described herein. In particular, prior to the present invention, there was no method known in the art for imaging mRNA. The method may comprise contacting the RNA, either in vivo or in a sample with the nanoparticles under conditions in which the RNA ligands present on the nanoparticles interact with target mRNA and detecting the nanoparticle-RNA-mRNA complex. The step of detecting the complex may be using an inherent property of the nanoparticles or by detecting a label associated with the nanoparticle. Preferred examples of labels suitable for use in this aspect of the invention include nanoparticles comprising magnetic groups, quantum dots or radionuclides. Quantum dots, for example as provided by nanocrystals of cadmium selenide, or other anions such as sulphide may be used in addition to selenide, have potential uses in biological imaging, in both electronic and optical devices, quantum computers and the screening of candidate drugs.

RNA-Nanopaticles as Tools in Functional Genomics

Genome screening normally utilises randomly generated RNA sequences which are then transfected into a test cell and changes in protein expression monitored. The nanoparticles of the present invention have two significant advantages over these prior art methods of genome screening. The first is that many of the random siRNA sequences will in fact have no effect, but at present the test cells need to be screened individually increasing the labour and cost of this form of these experiments. The present invention allows multiple siRNA sequences to be included as ligands on the nanoparticles, thereby providing the potential to speed up the rate of screening. Thus, if an effect is observed in a cell, the RNA molecules on present on a bead can then be screened to determine which of the sequence(s) were responsible for the effect. Furthermore, as the nanoparticles provide a delivery system for the RNA, the use of transfection agents required in the prior art may be avoided. This is a desirable advantage as the transfection agents may themselves cause changes in protein expression in the cells being tested. Therefore, in a further aspect, the nanoparticles of the present invention may be employed as tools in functional genomics, for example as described in Nature Reviews, Volume 3 April 2004, 318-329). The nanoparticles may have more than two, more than 5, more than 10 or more than 20 or more than 100 RNA sequences per particle to investigate gene function in vivo and also as a tool for genome-wide screening.

Aerosol Delivery

In a further aspect, the present invention provides the use of the nanoparticles described herein in aerosols. This is made possible by the small size of the nanoparticles. The aerosol compositions may be used to deliver RNA ligands, in particular to the lungs for imaging and/or therapeutic use, e.g. in the treatment of conditions affecting the lungs.

In any of the above aspects, the nanoparticles may be linked to therapeutically active substances such as antibodies or tumour-killing drugs. The magnetic properties of the nanoparticles can also be used to target tumours, by using a magnetic field to guide the nanoparticles to the tumour cells. However, use of magnetic field alone to direct nanoparticles to tumour cells is not always feasible or accurate, so the present invention provides an advantage by enabling the nanoparticles to be specifically directed to tumour cells via tumour-specific ligands. This will allow less drug to be used and reduce the chance of side effects, as the drug is directed only to the cells where it is needed and not to healthy cells.

Another advantage of the nanoparticles of the present invention is their exceptionally small size, which makes them more likely to be taken up by cells even when linked to targeting or therapeutic molecules.

In a further aspect, nanoparticles in which the ligand is an antigen can be administered as a vaccine, e.g. ballistically, using a delivery gun to accelerate their transdermal passage through the outer layer of the epidermis. The nanoparticles can then be taken up, e.g. by dendritic cells, which mature as they migrate through the lymphatic system, resulting in modulation of the immune response and vaccination against the antigen.

The nanoparticles of the invention may be formulated as pharmaceutical compositions that may be in the forms of solid of liquid compositions. Such compositions will generally comprise a carrier of some sort, for example a solid carrier such as gelatine or an adjuvant or an inert diluent, or a liquid carrier such as water, petroleum, animal or vegetable oils, mineral oil or synthetic oil. Physiological saline solution, or glycols such as ethylene glycol, propylene glycol or polyethylene glycol may be included. Such compositions and preparations generally contain at least 0.1 wt % of the compound.

The nanoparticle compositions may be administered to patients by any number of different routes. Parenteral administration includes administration by the following routes: intravenous, cutaneous or subcutaneous, nasal, intramuscular, intraocular, transepithelial, intraperitoneal and topical (including dermal, ocular, rectal, nasal, inhalation and aerosol), and rectal systemic routes. For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, solutions of the compounds or a derivative thereof, e.g. in physiological saline, a dispersion prepared with glycerol, liquid polyethylene glycol or oils.

In addition to one or more of the compounds, optionally in combination with other active ingredient, the compositions can comprise one or more of a pharmaceutically acceptable excipient, carrier, buffer, stabiliser, isotonicising agent, preservative or anti-oxidant or other materials well known to those skilled in the art. Such materials should be non-toxic and should not interfere with the efficacy of the active ingredient. The precise nature of the carrier or other material may depend on the route of administration, e.g. orally or parenterally.

Liquid pharmaceutical compositions are typically formulated to have a pH between about 3.0 and 9.0, more preferably between about 4.5 and 8.5 and still more preferably between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

Preservatives are generally included in pharmaceutical compositions to retard microbial growth, extending the shelf life of the compositions and allowing multiple use packaging. Examples of preservatives include phenol, meta-cresol, benzyl alcohol, para-hydroxybenzoic acid and its esters, methyl paraben, propyl paraben, benzalconium chloride and benzethonium chloride. Preservatives are typically employed in the range of about 0.1 to 1.0% (w/v).

Preferably, the pharmaceutically compositions are given to an individual in a prophylactically effective amount or a therapeutically effective amount (as the case may be, although prophylaxis may be considered therapy), this being sufficient to show benefit to the individual. Typically, this will be to cause a therapeutically useful activity providing benefit to the individual. The actual amount of the compounds administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 18th edition, Mack Publishing Company, Easton, Pa., 1990; and Handbook of Pharmaceutical Excipients, 2nd edition, 1994. By way of example, and the compositions are preferably administered to patients in dosages of between about 0.01 and 100 mg of active compound per kg of body weight, and more preferably between about 0.5 and 1 mg/kg of body weight.

Embodiments of the present invention will now be described by way of example and not limitation with reference to the accompanying figures.

### BRIEF DESCRIPTION OF THE FIGURES

FIG. 1 shows a transmission electron micrograph of RNA-Au-Glc nanoparticles.

FIG. 2 shows the testing for the presence of RNA in the prepared nanoparticles. (a) Without UV light: 1. RNA-Au-Glc nanoparticles+EtBr; 2. Glc-Au+EtBr; 3. Glc-Au; 4. residue of washing solution+EtBr. (b) With UV light: 1. RNA-Au-Glc nanoparticles+EtBr; 2. Glc-Au+EtBr; 3. Glc-Au; 4. residue of washing solution+EtBr.

FIG. 3a shows a Western blot of Her-2/neu protein from equal volumes of lysates of SKBR3 cells 48 hours after transfection with a Her-2/neu siRNA. C=Control (untreated) cells; Au=Cells treated with siRNA coupled to gold nanoparticles without RNAiFectamine; S=Silencing siRNA with RNAiFectamine; NS=Non-silencing siRNA with RNAiFectamine.

FIG. 3b shows a Western blot of Her-2/neu protein from equal volumes of OVCAR cell lysates 72 hours after transfection with a Her-2/neu siRNA. C=Control (untreated) cells; Au=Cells treated with siRNA coupled to gold nanoparticles without RNAiFectamine.

FIG. 4 shows a schematic representation of a preferred nanoparticle of the invention comprising siRNA and carbohydrate ligands.

FIG. 5 shows the effect on cell proliferation on OVCAR cells transfected with siRNA alone (A) or with siRNA-nanoparticles (B) at 0.25 μg (diamonds), 0.5 μg (squares), 1.0 μg (triangles), 1.5 μg (grey cross) and 2.0 μg (black cross) siRNA-nanoparticles per 1000 cells. X axis=days; Y axis=cell number (log₁₀)

FIG. 6 shows the effect on cell proliferation on OVCAR cells transfected with siRNA-nanoparticles with and without transfection reagent. Three concentrations of nanoparticles were used:

1: with transfection reagent (squares) and without transfection reagent (diamonds);

2: with transfection reagent (grey crosses) and without transfection reagent (triangles);

3: with transfection reagent (circles) and without transfection reagent (black crosses).

X axis=days; Y axis=cell number (log₁₀)

### DETAILED DESCRIPTION

### EXAMPLES

The Her-2/neu oncogene and its encoded product p185Her-2/neu belong to the epidermal growth factor receptor tyrosine kinases (Bargmann et al, 1986). The HER receptor family consists of four transmembrane tyrosine kinases: EGFR (also known as Her-1 or erbB-1), erbB-2 (Her-2), erbB-3 (Her-3), and erbB-4 (Her-4). Her-2/neu signalling pathways are known to play critical roles in cell growth and differentiation, malignant transformation, and resistance to chemotherapeutic agents (Yarden & Sliwkowski, 2001). Her-2/neu is over-expressed in about one third of cases of human breast or ovarian cancers, and its over-expression is associated with poor prognosis (Berchuck et al, 1990).

Numerous attempts have been made to inhibit Her-2/neu expression in cancer cells as a potential therapeutic approach. A humanized monoclonal antibody against Her-2/neu (Trastuzumab or Herceptin) has been effective in Her-2/neu-overexpressing metastatic cancer (Mendelsohn & Baselga, 2000; Baselga et al, 1996) but was found to up-regulate Her-3 expression. An antisense oligonucleotide against Her-2/neu has been shown to induce apoptosis in human breast cancer cell lines that overexpress Her-2/neu (Roh et al, 2000). Gene therapy with E1A, delivered by liposomes or by adenoviral vectors, can reduce mortality among tumour-bearing mice in a model of Her-2/neu-overexpressing ovarian cancer and can reduce the incidence of distant metastases in a model of breast cancer (Chang et al, 1996).

The down regulation of Her-2/neu expression was found to lead to decreases in PI3K, Akt, and phosphorylated Akt which resulted in decreased expression of cyclin D1, a cyclin involved in the regulation of G0/G1 cell arrest and oncogenic transformation (Sherr & Roberts, 1999). A recent study comparing the efficacy of antisense oligonucleotides and siRNA demonstrated that siRNAs are at least 10 times more efficient on a nM basis at silencing a reporter gene (Miyagishi et al, 2003). Several previous studies have demonstrated that Her-2/neu stimulates the transcription of VEGF, a potent proangiogenic factor (Kumar & Yarmand-Bagheri, 2001) the level of which was markedly decreased after silencing of Her-2/neu expression. Down regulation of Her-2/neu by retroviral siRNA increased thrombospondin-1 levels, a powerful inhibitor of angiogenesis (Izumi et al, 2002). In vitro data demonstrated that HER2 siRNA treatment also significantly up-regulates HLA class I surface expression in human tumours (Choudhury et al, 2004).

a. Strategy: Silencing

Her2/Neu cDNA Target Sequence:
AAG CCT CAC AGA GAT CTT GAA
a) Sense:
5′- G CCU CAC AGA GAU CUU GAAdTdT -3′
b) Antisense:
3′- dTdTC GGA GUG UCU CUA GAA CUU -5′
c) Sense:
5′- G CCU CAC AGA GAU CUU GAAdTdT -3′ SS
d) Antisense:
3′ SS-dTdTC GGA GUG UCU CUA GAA CUU -5′

b. Annealing

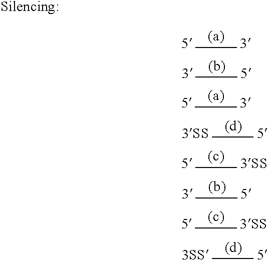

c. Possible GNP Combinations

| | | GNP | | |
|---|---|---|---|---|
| siRNA | Anneal | Glc- | Glcβ4GlcNAc | Glcβ4GlcNH₂ |
| Silencing | a-b | | | |
| | a-d | | | |
| | c-b | X | | |
| | c-d | | | |

X = used in this study.

d. Methods

1. Cell Lines

SK-BR-3 Human Mammary adenocarcinoma from ATCC (Cat.#HTB-30). OVCAR-3 Human ascites adenocarcinoma from NCI-Frederick Cancer DCTD Tumor/cell line repository (vial 0502296).

2. siRNA Stock Solutions

The Her-2/neu DNA target Sequence chosen was AAGCCTCACA GAGATCTTGAA.

The sense siRNA had a sequence r(GCCUCACAGAGAUCUUGAA) d(TT)3ThSS. (MW of K-salt 7416.25) and the antisense sequence r(UUCAAGAUCUCUGUGAGGC) d(TT)3ThSS (MW of K-salt 7409.57) were obtained from Qiagen.

The control (non-silencing) siRNA duplex sequences from Qiagen (Cat#1022076) where sense r(UUC UCC GAA CGU GUC ACG U)d(TT) and antisense r(ACG UGA CAC GUU CGG AGA A)d(TT) and the MW of the annealed K-salt was 14839.5.

Dissolve contents (296.65 μg) of one sense siRNA tube in 1 ml sterile buffer (100 mM potassium acetate, 30 mM Hepes-KOH, 2 mM magnesium acetate pH 7.4) to make a 40 μM stock. Each μl will contain 0.297 μg siRNA.

The contents (296.38 μg) of one antisense siRNA tube were dissolved in 1 ml sterile buffer (100 mM potassium acetate, 30 mM Hepes-KOH, 2 mM magnesium acetate pH 7.4) to make a 40 μM stock. Each μl contained 0.296 μg siRNA.

To anneal, 30 μl of each RNA oligo solution was combined with 15 μl of 5× annealing buffer. The final buffer concentration was 50 mM Tris, pH 7.5-8.0, 100 mM NaCl in DEPC-treated water. The final volume was 75 μl and the final concentration of siRNA duplex was 16 μM.

The solution was incubated for 1 minute in a water bath at 90-95° C., and allowed to cool to room temperature (i.e. below 30° C.). The tube was centrifuged briefly to collect all liquid at the bottom of the tube. Slow cooling to room temperature took 45-60 minutes. The resultant solution was stored at −20° C. until ready to use and was resistant to repeated freezing and thawing.

3. siRNA Nanogold Stock Solution

General Methods

HAuCl₄ (99.999%) and NaBH₄ were purchased from Aldrich Chemical Company. 2-thioethyl-β-D-glucopyranoside was synthesized in our laboratory using standard procedures. For all experiments and solutions, Nanopure water (18.1 mΩ) treated with DEPC (diethylpirocarbonate) was used. All eppendorfs, spatulas and vials were RNase free.

Annealed double-stranded siRNA was purchased from Qiagen-Xeragon Inc. The specifications were:

DNA target sequence
AAGCCTCACAGAGATCTTGAA.
Sense siRNA
r (GCCUCACAGAGACUUGAA) d (TT) 3′-Thiol-(SS)-C3-
linker on 3′
(MW of K-salt 7416.25)
Antisense
r (UUCAAGAUCUCUGUGAGGC) d (TT)
(MW of K-salt 7409.57)

e. Preparation of RNA-Au-Glc Nanoparticles

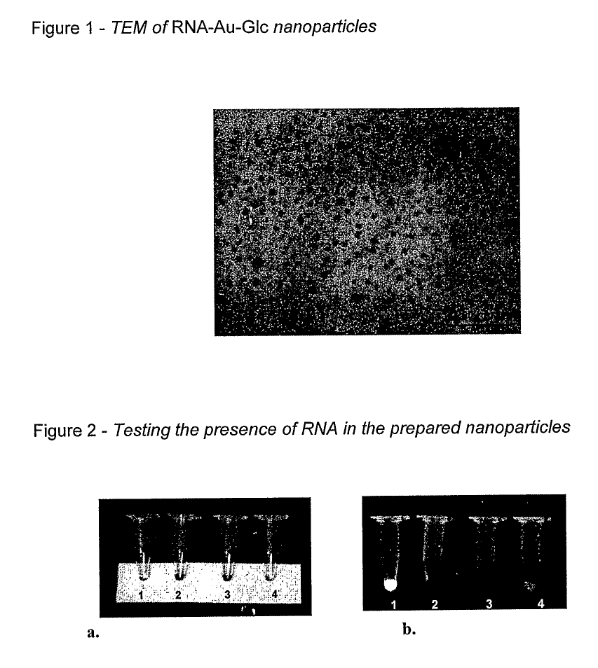

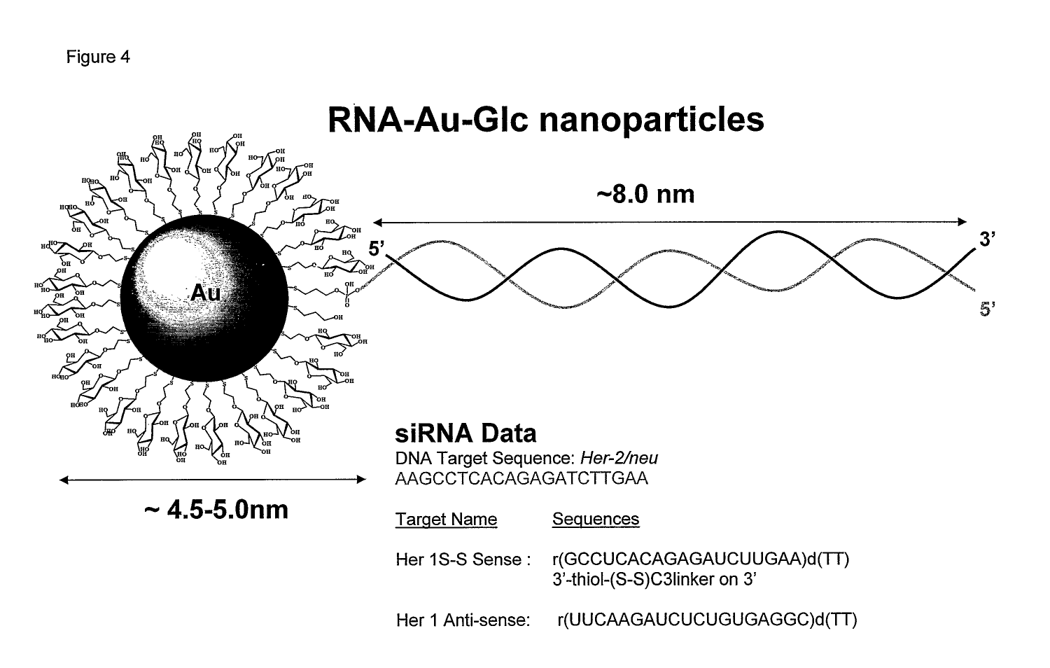

To a solution of 2-thioethyl-β-D-glucopyranoside (0.9 mg, 3.75 μmol) and siRNA (0.148 mg, 0.01 μmol) in TRIS buffer 100 mM, pH 7.7 (250 μL), an aqueous HAuCl₄ solution (22 μL, 0.025M) was added. Then, 1N aqueous solution of NaBH₄ (30 μL) was added in several portions with rapid shaking. The brown suspension formed was shaken for an additional 1 h at 4° C. The suspension was purified by centrifugal filtering (AMICON MW 10000, 30 min, 4° C., 14000 rpm). The process was repeated twice, washing with 125 μL of TRIS buffer. The residue in the AMICON filter was dissolved in 250 μL of TRIS buffer and lyophilised to afford 4 mg of RNA-Au-Glc nanoparticles (resuspension of the solid in 1 mL of water should give a 6±1 μM solution of RNA in 20 mM TRIS buffer). The filtrate was desalted using AMICON (MW 3000, 4° C., 14000 rpm) and lyophilised. The weight of the residue was <50 μg. The transmission electron micrograph (TEM) shown in FIG. 1 shows that the average size of the particles was 2.8 nm, with an average of 807 gold atoms/particle, siRNA and 100 molecules of the glucose derivative as represented in FIG. 4 and has an approximate MW>160,000.

f. Checking the Presence of RNA in Nanoparticles

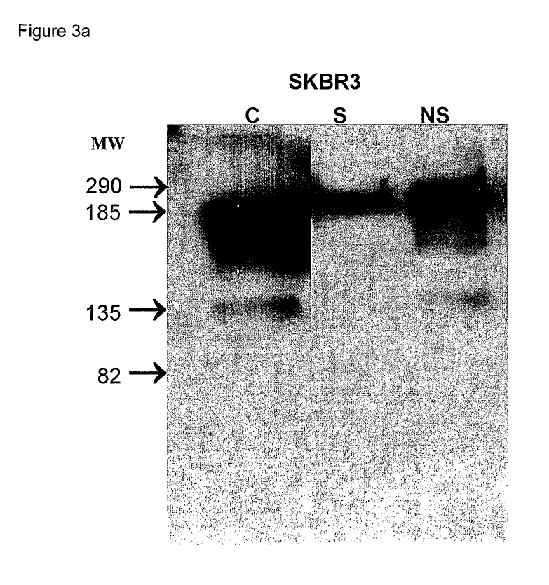

RNA-Au-Glc nanoparticles, Glc-Au nanoparticles and the residue of washing the RNA-Au-Glc nanoparticles which presumably containing RNA oligonucleotide and glucose derivative were each dissolved in 30 μL of water. Aliquots of these solutions (1 μL) were mixed with aqueous solution of ethidium bromide (EtBr) (1 μL, 0.1% v/v). Fluorescence was observed under a UV lamp (see FIG. 2) demonstrating that the so-prepared nanoparticles have incorporated siRNA (FIG. 2b, tube 1), while the nanoparticles containing only glucose do not show any fluorescence (FIG. 2b, tube 2).

4 mg of the siRNA/nanogold complex generated from 148 μg siRNA after were dissolved in 1 ml water to obtain a 6±1 μM stock solution in 20 mM tris. Each μl of solution contained the equivalent of 0.078 μg siRNA.

Cell Plating

1. 24 h before transfection, 6×10⁴ cells were pipetted into a 24-well plate and the volume made up to 0.5 ml with appropriate culture medium.

2. The cells were allowed to reach 50-80% confluency, taking approximately 24 h.

3. The culture medium was removed and replaced with 300 μl fresh medium/well.

Transfecting Cells with siRNA Complex

1. 3.3 μl (or 12.8 μl of the nanogold complex) of the appropriate duplex siRNA stock was dispensed into a 24-well plate corresponding to that with cells.

2. To each well 96.7 μl (or 87.2 μl for the nanogold complex) of the appropriate culture medium was added and mixed well by pipetting up and down 5 times.

3. To each well (other than the nanogold complex wells) 6 μl of RNAiFect was added and mixed well by pipetting up and down 5 times.

4. The solutions were incubated at room temperature for 10-15 minutes to allow complex formation.

5. The cells in 300 μl culture medium were overlayed with the 100 μl of the appropriate transfection complex.

6. The plate gently rocked to mix avoiding swirling.

7. The plate was incubated at 37° C. in a CO₂ incubator for 48-72 h.

8. The medium was removed and the cells washed three times with ice cold PBS.

9. The cells were lysed and the protein content of the lysates determined.

10. The proteins were separated by SDS-PAGE followed by Western blotting using a Her2/ErbB2 polyclonal rabbit antibody from Cell Signalling Technology (Cat.#2242).

11. The blots were treated with an anti-rabbit IgG-HRP conjugate followed by ECL development.

g. Results

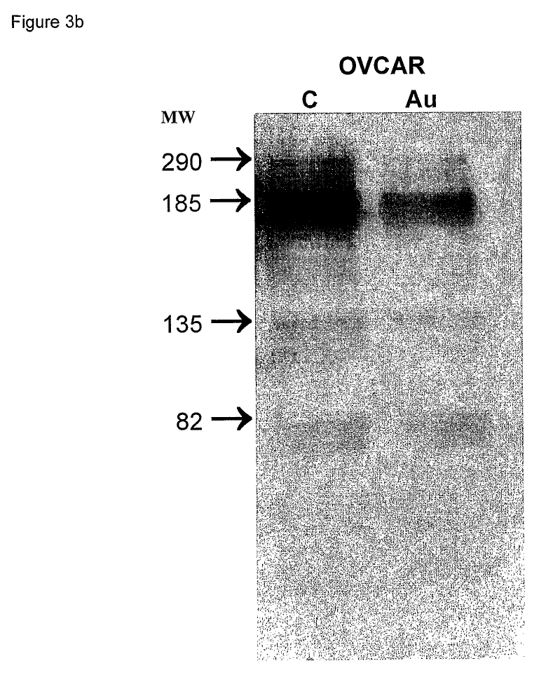

Preliminary observations using 1 μg siRNA/well are shown in FIGS. 3a and 3b. siRNA-gold nanoparticles were added to cells without RNAiFectamine. The SKBR3 cells were slower to reach 80% confluency than the OVCAR cells. The SKBR3 results are from lysates 48 h after transfection while the OVCAR results were from lysates 72 h after transfection. A schematic of the nanoparticles are shown in FIG. 4.

Non-Toxicity of siRNA-Au-Glc Nanoparticles to Cells

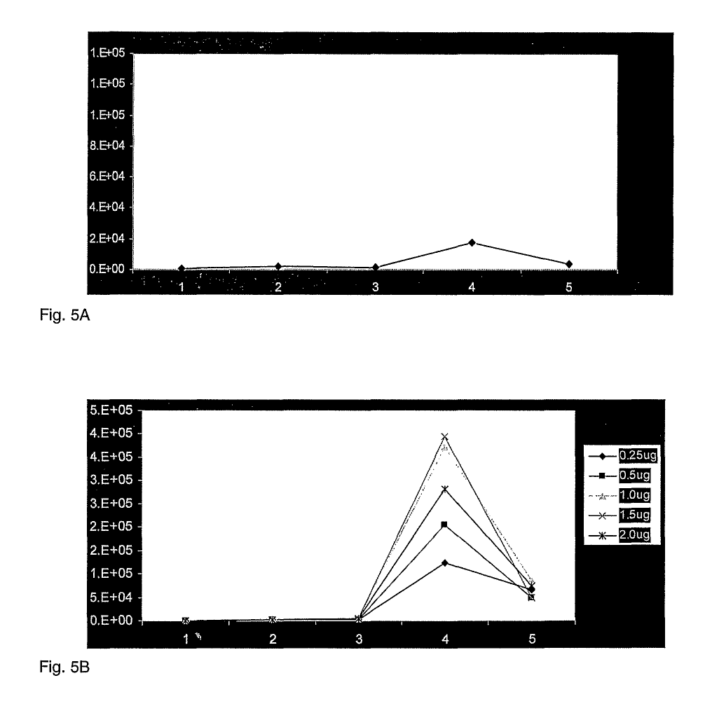

Cells were transfected with siRNA alone and with siRNA conjugated to gold glyconanoparticles. FIG. 5 shows that the nanoparticle-conjugated siRNA was effective and had no toxic effects. A dose-dependent effect on cell number was seen indicating that the siRNA nanoparticles increased cell proliferation.

Entry of siRNA-Au-Glc Nanoparticles Into Cells

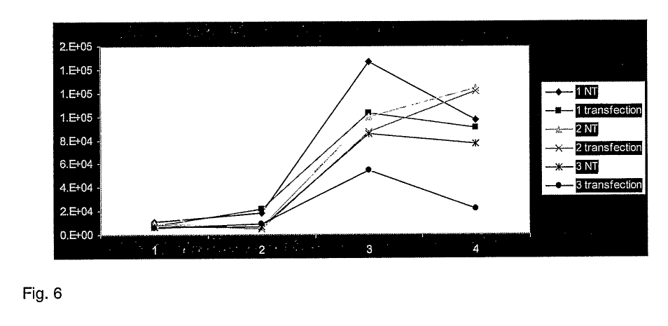

OVCAR cells were transfected with siRNA-nanoparticles with and without the transfection reagent usually required for transfection of cells with siRNA. FIG. 6 shows that the transfection reagent was not required for entry of siRNA-nanoparticles into the cells. The results show that the siRNA nanoparticles were effectively delivered into the cells even in the absence of transfection reagent; indeed, delivery appeared to be more efficient without transfection reagent. The dose-dependency of the effect on cell number indicates a genuine response to the siRNA-nanoparticles.

### REFERENCES

The references mentioned herein are all expressly incorporated by reference.

Pal-Bhadra et al. RNAi-Mediated targeting of Heterochromatin by the RITS complex. Science (2004) vol. 303, 669.

Verdel et al. Heterochromatic silencing and HP1 localization in Drosphila are dependent on the RNAi Machinery. Science (2004) vol. 303, 672.

Elbashir et al. Duplexes of 21-nucleotide RNAs mediate RNA interference in cultured mammalian cells. Nature 2001;411:494-8.

Tuschl. RNA interference and small interfering RNAs. Chem Biochem 2001;2:239-45.

Bargmann et al. The neu oncogene encodes an epidermal growth factor receptor-related protein. (1986) Nature 319, 226-230.

Yarden & Sliwkowski. Untangling the ErbB signalling network. (2001) Nat Rev Mol Cell Biol 2, 127-137.

Berchuck et al. Overexpression of HER-2/neu is associated with poor survival in advanced epithelial ovarian cancer. (1990) Cancer Res 50, 4087-4091.

Mendelsohn & Baselga. The EGF receptor family as targets for cancer therapy. (2000) Oncogene 19, 6550-6565.

Baselga et al. Phase II study of weekly intravenous recombinant humanized anti-p185HER2 monoclonal antibody in patients with HER2/neu over expressing metastatic breast cancer. J Clin Oncol 1996;14:737-44.

Roh et al. Down regulation of HER2/neu expression induces apoptosis in human cancer cells that over-express HER2/neu. (2000) Cancer Res 60, 560-565.

Chang et al. Inhibition of intratracheal lung cancer development by systemic delivery of E1A. (1996) Oncogene 13, 1405-1412.

Sherr & Roberts. CDK inhibitors: positive and negative regulators of G1-phase progression. (1999) Genes Dev 13, 1501-1512.

Miyagishi et al. Comparison of the suppressive effects of antisense oligonucleotides and siRNAs directed against the same targets in mammalian cells. Antisense Nucleic Acid Drug Dev 2003;13:1-7.

Kumar & Yarmand-Bagheri. The role of HER2 in angiogenesis. (2001) Semin Oncol 28, 27-32.

Izumi et al. Tumour biology: herceptin acts as an antiangiogenic cocktail. (2002) Nature 416, 279-280.

Choudhury et al. Small interfering RNA (siRNA) inhibits the expression of the Her2/Neu gene, upregulates HLA Class I and induces apoptosis of Her2/Neu positive tumour cell lines. Int J Cancer 108, 71-77, 2004.

Overbaugh, HTLV sweet-talks its way into cells, Nat. Med (2004) vol. 10, 20.

Check. RNA to the rescue. Nature (2003) vol 425, 10-12.

Zamore et al. siRNAs knock down hepatitis. Nature (2003) vol 9, 266-267.

Song et al. RNA interference targeting Fas protects mice from fulminant hepatitis. Nat. Med. (2003) vol. 9, 347-351.

Matzke & Matzke. RNAi Extends its Reach. Science (2003) Vol 301, 1060-1061.

WO 02/32404

PCT application claiming priority from GB-A-0313259.4.

1. A nanoparticle which comprises a core including metal and/or semiconductor atoms, wherein the core is covalently linked to a plurality of ligands and the ligand comprise a RNA ligand.

2. The nanoparticle of claim 1, wherein the RNA ligands are siRNA or miRNA ligands.

3. The nanoparticle of claim 1, wherein the nanoparticles further comprises ligands which include carbohydrate groups.

4. The nanoparticle of claim 2, wherein the nanoparticle is covalently linked to a siRNA molecule via a linker group.

5. The nanoparticle of claim 4, wherein the linker group is a thiol group, an ethylene group or a peptide group.

6. The nanoparticle of claim 1, wherein the RNA ligands are between 17 and 30 ribonucleotides in length.

7. The nanoparticle of claim 1, wherein the ligand is a siRNA ligand and comprises a 3′ overhang of 2 ribonucleotides.

8. The nanoparticle of claim 1, wherein a first sense or antisense strand of a RNA molecule is covalently linked to a nanoparticle core via its 5′ and/or 3′ end.

9. The nanoparticle of claim 8, wherein a second strand of the RNA molecule which is complementary to the first strand is annealed to the first strand of the RNA molecule.

10. The nanoparticle of claim 9, wherein the second RNA strand is covalently linked to a nanoparticle core via its 5′ and/or 3′ end.

11. The nanoparticle of claim 9, wherein the first and second strands of the RNA molecule are separately linked to nanoparticle cores and subsequently annealed together.

12. The nanoparticle of claim 1, wherein the RNA ligand is a miRNA ligand and comprises a hairpin.

13. The nanoparticle of claim 1, wherein the RNA ligand is based on a Her2 gene sequence.

14. The nanoparticle of claim 1, wherein the nanoparticle comprises a label.

15. The nanoparticle of claim 14, wherein the label is a fluorescent group, a radionuclide, a magnetic label, a dye, a NMR active atom, or an atom which is capable of detection using surface plasmon resonance.

16. The nanoparticle of claim 15, wherein the fluorescent group is fluorescein, rhodamine or tetramethyl rhodamine, Texas-Red, Cy3, or Cy5.

17. The nanoparticle of claim 15, wherein the radionuclide is ^{99m}Tc, ³²P, ³³P, ⁵⁷Co, ⁵⁹Fe³⁺, ⁶⁷Cu²⁺, ⁶⁷Ga³⁺, ⁶⁸Ge, ⁸²Sr, ⁹⁹Mo, ¹⁰³Pd, ¹¹¹In³⁺, ¹²⁵I, ¹³¹I, ¹³⁷Cs, ¹⁵³Gd, ¹⁵³Sm, ¹⁵⁸Au, ¹⁸⁶Re, ²⁰¹Tl⁺, ³⁹Y³⁺, ⁷¹Lu³⁺ or ²⁴Cr²⁺.

18. The nanoparticle of claim 15, wherein the magnetic label is a paramagnetic group comprising Mn⁺², Gd⁺³, Eu⁺², Cu⁺², V⁺², Co⁺², Ni⁺², Fe⁺², Fe⁺³, or lanthanides⁺³.

19. The nanoparticle of claim 15, wherein the NMR active atom is Mn⁺², Gd⁺³, Eu⁺², Cu⁺², V⁺², Co⁺², Ni⁺², Fe⁺², Fe⁺³ or lanthanides⁺³.

20. The nanoparticle of claim 1, wherein the nanoparticle is water soluble.

21. The nanoparticle of claim 1, wherein the core of the nanoparticle has a mean diameter between 0.5 and 10 nm.

22. The nanoparticle of claim 1, wherein the core of the nanoparticle has a mean diameter between 1 and 5 nm.

23. The nanoparticle of claim 1, wherein the nanoparticle including its ligands has a mean diameter between 10 and 30 nm.

24. The nanoparticle of claim 1, wherein the core is a metallic core.

25. The nanoparticle of claim 24, wherein the metallic core comprises Au, Ag or Cu.

26. The nanoparticle of claim 24, wherein the metallic core is an alloy selected from Au/Ag, Au/Cu, Au/Ag/Cu, Au/Pt, Au/Pd, Au/Ag/Cu/Pd, Au/Fe, Au/Cu, Au/Gd, Au/Fe/Cu, Au/Fe/Gd or Au/Fe/Cu/Gd.

27. The nanoparticle of claim 24, wherein the core of the nanoparticle is magnetic.

28. The nanoparticles of claim 24, wherein nanoparticle comprises passive metal atoms and magnetic metal atoms in the core in a ratio between about 5:0.1 and about 2:5.

29. The nanoparticle of claim 28, wherein the passive metal is gold, platinum, silver or copper, and the magnetic metal is iron or cobalt.

30. The nanoparticle of claim 27, wherein the core comprises MnFe (spinel ferrite) or CoFe (cobalt ferrite).

31. The nanoparticle of claim 1, wherein the core comprises of semiconductor atoms.

32. The nanoparticle of claim 31, wherein the semiconductor atoms are capable of acting as a quantum dot.

33. The nanoparticle of claim 31, wherein the semiconductor is cadmium selenide, cadmium sulphide, cadmium tellurium or zinc sulphide.

34. The nanoparticle of claim 1, wherein the nanoparticle comprises a plurality of different types of ligand.

35. The nanoparticle of claim 34, wherein the ligands further comprise a peptide, a protein domain, a nucleic acid segment or a carbohydrate group.

36. The nanoparticle of claim 34, wherein the ligands comprise a polysaccharide, an oligosaccharide or a monosaccharide group.

37. The nanoparticle of claim 34, wherein the ligand is a glyconanoconjugate.

38. The nanoparticle of claim 37, wherein the glyconanoconjugate comprises a glycolipid or a glycoprotein.

39. The nanoparticle of claim 34, wherein the ligands comprise DNA or RNA.

40. A composition comprising a population of one or more of the nanoparticles of claim 1.

41. A method of preparing nanoparticles of by conjugating RNA to the core of a nanoparticle, the method comprising:
derivatising the first and/or second strand of the RNA with a linker to produce first and/or second RNA strand derivatised with the linker at the 5′ and/or 3′ ends; and
reacting the linker derivatised RNA with reactants for producing the core of the nanoparticle so that during self-assembly of the nanoparticles, the nanoparticle cores attach to the RNA via the linker.

42. The method of claim 41, wherein the linker is a thiol linker, an ethylene linker or a peptide linker.

43. The method of claim 41, wherein the reaction mixture comprises derivatised siRNA, a salt of at least one of metal atoms and semiconductor atoms and a reducing agent to produce the nanoparticles.

44. Nanoparticles as obtained by the method of claim 41.

45.-54. (canceled)

55. A method of use of down regulating a target gene, the method comprising contacting cells containing the gene with nanoparticles comprising a core including metal and/or semiconductor atoms, wherein the core is covalently linked to a plurality of ligands and the ligand comprise a RNA ligand.

56. The method of claim 55, wherein the method is carried out in vitro.

57. The method of claim 55, wherein the method produces a transient knock out of the target gene.

58. The method of claim 55, wherein the method employs nanoparticles having at least two different RNA ligands, either conjugated to the same nanoparticles or present in a composition of at least two different types of nanoparticles, to down regulate expression in at least two genes in a pathway.

59. The method of claim 58, wherein the pathway is an inflammatory pathway, an anti-viral pathway, a signalling pathway in cancer, a metastasis pathway or a metabolic pathway.

60. The method of claim 55, wherein the RNA ligand is based on a conserved domain of a gene family to down regulate the expression of a plurality of members of the gene family.

61. (canceled)

62. A method for detecting and/or imaging mRNA employing nanoparticles of claim 1, the method comprising contacting the nanoparticles with a sample containing target mRNA under conditions in which the RNA ligands present on the nanoparticles are capable of interacting with target mRNA and detecting the nanoparticle-RNA-mRNA complex.

63. The method of claim 62, wherein the step of detecting the complex uses an inherent property of the nanoparticles or by detecting a label associated with the nanoparticle.

64. The method of claim 63, wherein the labels are magnetic groups, quantum dots or radionuclides.

65. The method of claim 62, wherein the method is carried out in vitro on a sample containing the mRNA.

66. The method of claim 62, wherein the method is carried out in vivo for detecting mRNA in a cell-containing sample.

67.-70. (canceled)

71. A method of treatment of a condition ameliorated by the down regulation of expression of a gene or a condition associated with over expression of a gene in a patient in need of said treatment by administering nanoparticles comprising a core including metal and/or semiconductor atoms, wherein the core is covalently linked to a plurality of ligands and the ligand comprise a RNA ligand, said nanoparticles being administered in an amount effective to cause down regulation of said gene by said RNA ligand.

72. The method of claim 71, wherein said nanoparticles are administered for the treatment of cancer, viral infection or macular degeneration of the eye.

73. The method of claim 72, wherein the cancer is breast cancer or the virus is HIV, hepatitis or influenza.

74. The method of claim 72, wherein the RNA is based on the Her2/Neu gene sequence.

75. The method of claim 72, wherein the nanoparticles comprise a further ligand conjugated to the core of the nanoparticle or a domain associated with the RNA molecule, wherein the further ligand or domain is effective to interact specifically with cells expressing said gene.

76. The method of claim 74, wherein the further ligand or domain is a member of a specific binding pair that is effective to bind specifically to its binding partner present on the surface of or inside of said cells.

77. The method of claim 71, wherein the RNA molecule targets the nanoparticles to cells which express mRNA that interacts with the RNA molecule.

78. The method of claim 78, wherein the nanoparticles further comprise a radionuclide, drug or other agent for treating or killing the cells targeted by the RNA molecule.

79. The method of claim 78, wherein the nanopaticles comprise a membrane translocation signal so that they are capable of permeating through the cell membrane.

80. The method of claim 71, wherein said nanoparticles are administered in the form of an aerosol and delivered to the lungs, and the nanoparticles comprise a label for imaging a condition affecting the lungs of a mammal.

81. The method of claim 71, wherein said nanoparticles are administered in the form of an aerosol and delivered to the lungs for the treatment of a condition affecting the lungs of a mammal.


SEQUENCE LISTING (5 sequences)

SEQ ID NO: 1 - DNA, 21 bp, Artificial Sequence
  Synthetic Sequence
aagcctcacagagatcttgaa

SEQ ID NO: 2 - DNA, 21 bp, Artificial Sequence
  Synthetic Sequence
gccucacagagaucuugaatt

SEQ ID NO: 3 - DNA, 21 bp, Artificial Sequence
  Synthetic Sequence
uucaagaucucugugaggctt

SEQ ID NO: 4 - DNA, 21 bp, Artificial Sequence
  Synthetic Sequence
uucuccgaacgugucacgutt

SEQ ID NO: 5 - DNA, 21 bp, Artificial Sequence
  Synthetic Sequence
acgugacacguucggagaatt